(19) United States
(12) Patent Application Publication
(10) Pub. No.: US 2003/0010338 A1
(43) Pub. Date: Jan. 16, 2003

(54) Particle flow control onto chuck

(75) Inventor: Thomas Nilsson, Mariefred (SE)

Correspondence Address: YOUNG & THOMPSON, 745 SOUTH 23RD STREET 2ND FLOOR, ARLINGTON, VA, 22202

(73) Assignee: MICRODRUG AG

(21) Appl. No.: 09/929,358

(22) Filed: Aug. 15, 2001

(30) Foreign Application Priority Data
Jul. 13, 2001 (SE) 0102521-2

(51) Int. Cl. A61M 15/00

(52) U.S. Cl. 128/203.15

(57) ABSTRACT

A method and a device involving an electric iris diaphragm/shutter for controlling particle transfer of electrically charged medication powder particles from a source to a defined target area or areas, of a chuck member. Spatial distribution of particles onto the target area or areas is achieved by an electro-dynamic field technique applied to the distribution and deposition of particles in a dose forming process. An electric iris diaphragm/shutter is located between a particle generator and the electrostatic chuck member such that all particles must pass the iris diaphragm for being transferred to the electrostatic chuck. By adjusting amplitude and frequency of a superimposed AC potential charged particles will oscillate in the created AC field such that only small light particles will emerge from the iris diaphragm/shutter for further transfer in the dose forming process.

### TECHNICAL FIELD

The present invention relates to a method and a device for controlling the flow and spatial distribution of dry, electrically charged medication powder being deposited on pre-defined areas of an electrostatic chuck in a dose forming process, and more specifically by using an electric iris diaphragm/shutter in forming pre-metered doses particularly of finely divided dry medication electro-powder.

### BACKGROUND

The dosing of drugs is carried out in a number of different ways in the medical service today. Within health care there is a rapidly growing interest in the possibility of dosing systemic acting medication drugs as a powder directly to the airways and lungs of a patient by means of an inhaler in order to obtain an effective, quick and user-friendly administration of such substances.

A dry powder inhaler, DPI, represents a device intended for administration of powder into the deep or upper lung airways by oral inhalation. A deep lung deposition for systemic delivery of medication drugs, but for local treatment of the airways the objective is local deposition, not deep lung. With deep lung should be understood the peripheral lung and alveoli, where direct transport of active substance to the blood can take place. For a particle in order to reach into the deep lung the aerodynamic particle size should typically be less than 3 μm, and for a local lung delivery typically less than 5 μm. Larger particle sizes will easily stick in the mouth and throat, which underlines the importance of keeping the particle size distribution of the dose within tight limits to ensure that a high percentage of the dose actually is deposited in the deep lung upon inhalation when the objective is systemic delivery of a drug. Furthermore, the inspiration must take place in a calm manner to decrease air speed and thereby reduce deposition in the upper respiratory tracts.

To succeed with systemic delivery of medication powders to the deep lung by inhalation there are some criteria, which have to be fulfilled. It is for instance very important to obtain a high dosing accuracy in each administration to the user. A very high degree of de-agglomeration of the medication powder is also of great importance. This is not possible with dry powder inhalers of today without special arrangements as for example a so-called spacer.

Powders for inhalers have a tendency of agglomerating, in other words to clod or to form smaller or larger lumps, which then have to be de-agglomerated. De-agglomeration is defined as breaking up agglomerated powder by introducing electrical, mechanical, or aerodynamic energy. Usually de-agglomeration is performed in at least two stages: stage one is in the process of depositing powder while building up the dose and stage two is in the process of dispersing the powder during the patient's inspiration of air through the DPI.

The term electro-powder refers to a finely divided medication powder presenting controlled electric properties being suitable for administration by means of an inhaler device. Such an electro-powder provides possibilities for a better dosing from equipment using a technique for electric field control such as disclosed in our U.S. Pat. No. 6,089,227 as well as our Swedish Patents No. 9802648-7 and 9802649-5, which present excellent inhalation dosing performance. The state of the art also discloses a number of solutions for depositing powder for dosing. The International Application WO 00/22722 presents an electrostatic sensing chuck using area matched electrodes. U.S. Pat. No. 6,063,194 discloses a powder deposition apparatus for depositing grains on a substrate using an electrostatic chuck having one or more collection zones and using an optical detection for quantifying the amount of grains deposited. U.S. Pat. No. 5,714,007 and U.S. Pat. No. 6,007,630 disclose an apparatus for electrostatically depositing a medication powder upon predefined regions of a substrate, the substrates being used to fabricate suppositories, inhalants, tablet capsules and the like. In U.S. Pat. No. 5,699,649 and U.S. Pat. No. 5,960,609 are presented metering and packaging methods and devices for pharmaceuticals and drugs, the methods using electrostatic photo technology to package microgram quantities of fine powders in discrete capsule and tablet form.

When using electrostatic technology and/or electrical fields in combination with electrostatic charging of the powder particles in a deposition process, a common difficulty encountered is to remove or neutralize the charge of the particles and the charge of the dose carrier, if an isolator, when the particles are being deposited on the carrier during the forming of the dose. If the removal of charges is incomplete or takes too long it will affect the forming of the dose negatively in that the charged particles already deposited will present a local repelling electric field, which tends to stop newly attracted particles from settling onto the targeted area of the substrate and forces newcomers to settle at the outskirts of the target area or areas. As more particles are deposited on the target area or areas the repelling field grows in strength. Finally, the field will be so strong that further deposition is not possible even if the net field strength at some distance from the target area or areas is exerting an attractive force on the charged particles.

In cases where electrostatic chucks are used, regardless of whether the chuck member, normally of a dielectric material, is pre-charged in the deposition area or areas to create the necessary local electric field in the target area(s), or a system of electrodes are used to attract the charged particles or if a combination of pre-charging and electrodes are used, it is always difficult to fill the target area or areas with the correct amount of particles. This is partly because the repelling field grows stronger with every particle deposited, leading to a spreading out of particles over a larger area than the intended target area, partly because of errors introduced by ambient particles e.g. water vapor, dust and ions, which are also electrostatically attracted to the target areas. Often, the chuck principle also requires powders of predetermined or known specific charge (μC/g) in order to predict the mass of particles attracted to the chuck, which in itself presents a big challenge. The answer to this problem is to introduce on-line measuring means of the quantity of the accumulated particles as they are deposited. This may require the chuck to be provided with deposition electrodes, shield electrodes, backing electrodes and sensing electrodes and control systems for measuring and adjusting the net charge of the respective target area in order to improve the quality of the transfer, distribution and deposition of the charged particles and the measuring of the resulting powder dose. The target area or areas, i.e. the deposition area(s), sometimes being beads, which are captured and held by the chuck for instance by electrostatic force during the deposition of particles onto the beads themselves. For reasons mentioned it is often impossible to form doses of sufficient mass and suitable spatial shape on the intended target or carrier.

Further, prior art technology devices seldom reach a sufficiently high degree of de-agglomeration, and an exact dose with a low relative standard deviation (RSD) between doses is not well controlled. This is partly due to difficulties in controlling the production line parameters during production of the doses, partly to shortcomings in the design of the inhaler device, which makes it hard to comply with regulatory demands. The difficulties leave much to be desired when it comes to dose conformity and lung deposition effectiveness of the medication substance. Therefore, there is still a demand for prefabricated high accuracy pre-metered doses to be loaded into an inhaler device, which then will ensure repeated and exact systemic or local pulmonary delivery of doses administered by inhalation.

### SUMMARY

A method and a device are defined for controlling the transfer of charged particles of a medication powder emitted from a particle generator to one or more defined target areas of an electrostatic chuck member in a dose forming process. One or more particle transfer electrodes are arranged between the chuck and the generator to form an electric iris diaphragm and shutter with electric fields associated for the transfer of the powder particles from the particle generator to the defined target areas of the electrostatic 10 chuck. Each target area is arranged to carry a pre-metered powder dose, the electric iris diaphragm and shutter will control the direction and speed of particles in the dose forming process. Either the dose is formed directly on the respective target area of the chuck or indirectly if the target area holds intermediate receivers, e.g. beads, which later may or may not be separated from the medicament powder dose. The electric iris diaphragm/shutter is located between the particle generator and the electric iris diaphragm and shutter such that all particles must pass the iris diaphragm to be transferred to the chuck. This iris diaphragm is also operating as a shutter. By adjusting amplitude and frequency of a superimposed AC potential, charged particles will oscillate in the created AC field such that only small light particles emerge from the iris diaphragm/shutter for further transfer in the dose forming process. In a preferred embodiment the process operates in an upward direction, i.e. against gravitation forces to prevent particles having no charge reaching the dose carrier in an uncontrolled way. Furthermore by the adjustment of amplitude and frequency a majority of charged particles emerging are accelerated and retarded in synchronism with the AC field, such that they impact on a defined target area or areas of the chuck member with a low speed and momentum resulting in a desired dose porosity.

The method according to the present invention is set forth by the independent claims 1, 11 and 12 and further embodiments of the method are set forth by the dependent claims 2 to 10.

A particle transfer control device is set forth by the independent claim 13 and further embodiments are defined by the dependent claims 14 to 20.

### BRIEF DESCRIPTION OF THE DRAWINGS

The invention, together with further objects and advantages thereof, may best be understood by referring to the following detailed description taken together with the accompanying drawings, in which:

FIG. 1 displays in principle a first embodiment of an electric iris diaphragm/shutter using one electrode only, showing charged particles as they are being transferred from the particle generator to one of the target areas of the electrostatic chuck;

FIG. 2 displays the same embodiment as in FIG. 1 but with the transfer of particles inhibited by a repelling acting electric field emanating from the electrode of the iris diaphragm/shutter;

FIG. 3 displays in principle a second embodiment of an electric iris diaphragm/shutter using two electrodes, showing charged particles as they are being transferred from the particle generator to one of the target areas of the electrostatic chuck;

FIG. 4 displays in principle a typical embodiment of an electric iris diaphragm/shutter using two electrodes and a wafer type insulator;

FIG. 5 displays in principle a third embodiment of an iris diaphragm using four electrodes, showing charged particles as they are transferred from the particle generator to one of the target areas of the electrostatic chuck, which may be repositioned by a servo mechanism as a part of the dose forming process;

FIG. 6 displays in principle one side of a typical iris diaphragm showing a second electrode;

FIG. 7 displays in principle one side of a typical iris diaphragm showing a first electrode;

FIG. 8 displays in principle an iris diaphragm using two electrodes, a dose being formed onto one of the target areas of the electrostatic chuck and two ion sources for removing accumulated charge in the dose being formed;

FIG. 9 displays in principle an iris diaphragm using two electrodes, a dose being formed onto one of the target areas of the electrostatic chuck, a servo arrangement for positioning the electrostatic chuck in relation to the iris and an ion source for neutralizing accumulated charge in the dose being formed;

FIG. 10 displays schematically an electrostatic chuck, an iris diaphragm, a dose in forming and an ion source positioned behind the electrostatic chuck connecting without physical contact the third voltage source with the third electrode; and

FIG. 11 is a flow diagram illustrating the method of the present invention.

### DESCRIPTION OF THE INVENTION

The present invention discloses a method and a device involving an electric iris diaphragm for controlling the particle transfer of electrically charged medication powder particles from a source to one or more defined areas, the target area or areas, of an electrostatic chuck. Spatial distribution of particles onto the target area or areas or dose bed(s) is achieved by means of electro-dynamic field technique applied to the distribution and deposition of particles in a dose forming process. The term “electro-dynamic field technique” in the context of this document refers to the effective electric field in four dimensions, space and time, resulting from well controlled, in terms of timing, frequency and amplitude, potentials applied to a number of electrodes placed in suitable positions in the space confined by a dose forming apparatus. The term “quasi-stationary electric field” in this context is used to describe an electric field or fields being controlled by voltage source devices having controlled impedances, all part of a control system, in which the applied voltages may be controlled arbitrarily and individually in the low-frequency time-domain. To facilitate the understanding of where and how voltages are applied all voltages are assumed to be referenced to ground potential throughout this document. Ground potential may of course be exchanged for an arbitrary potential when utilizing the invention. It will be apparent to a person skilled in the art that any singular potential or voltage may be referenced to another potential or voltage source, e.g. in order to simplify or improve a control system, without departing from the spirit and scope of the invention as defined by the appended claims.

A particle generator produces positively and/or negatively charged particles by corona-, tribo- or induction-charging. The charged particles are emitted from the generator into a controlled atmosphere, normally air, where they enter an electric field coming from suitably positioned electrodes given suitable potentials by suitable voltage sources and controlled circuit impedance. At least one of the electrodes comprises an electric iris diaphragm/shutter. The iris diaphragm/shutter has at least one aperture of suitable size and shape where particles can pass through and it is positioned between the particle generator and the electrostatic chuck. The strength and direction of the composed electric field between the particle generator and the iris diaphragm depends on the size and shape of the electrodes used, their relative positions and not least on the potentials applied to the electrode or electrodes of the iris diaphragm as well as to the other electrodes. In this way, it is possible to control the electric forces acting on the charged particles, which are attracted to or repelled from parts or all of the iris diaphragm and its apertures. Charged particles passing through an aperture of the iris diaphragm are attracted by the oppositely charged target area or areas of the chuck if pre-charging by e.g. the corona method is used. Alternatively, the particles enter a further applied electric field set up between ground, or any other electric reference, and an electrode supplied with a potential from a voltage source. The electrode is preferably positioned behind the target area or areas of the chuck and it is either common to all areas, or the electrode may be individual to each target area or the target areas may be divided up between a smaller number of electrodes. Areas of the chuck which are not target areas may be protected against particle deposition by shield electrodes or a ground plane integrated in the chuck member and given a potential of opposite polarity repelling the charged particles. Provided that the relative positions of the iris apertures and the target areas are reasonably aligned, the charged particles leaving the iris diaphragm at this stage are captured by the field and attracted to the chuck so they begin to travel in that direction along the field lines until they hit the target area or areas of the chuck where they are deposited.

Two properties of the iris diaphragm/shutter are of particular importance. The first one is the ability to control the apparent size of the aperture or apertures of the electric iris diaphragm such that it appears smaller or larger to the attracted particles depending on what voltage potentials are applied to the electrodes. This opens the possibility to control the area of particle flow through the iris diaphragm and consequently the utilized area of the target area or areas of the chuck member onto which the transported particles will be deposited. The second important property is that the electric iris diaphragm can be made to work as a particle flow control valve, i.e. a shutter arrangement, such that it is possible to switch the flow of particles completely on or off by simply feeding suitable voltages to the electrodes, which will turn the composite electric field in the opposite direction then forcing charged particles away from the iris diaphragm. In fact, by adjusting the voltages suitably, it is also possible to partly control the amount of particles per unit time that are let through and in this manner trim the particle deposition rate on the target area or areas. In a preferred embodiment, however, the iris diaphragm is mainly used for area size control and switching the flow on or off instantly.

The potentials applied to the electrodes of the iris diaphragm are controlled by a control system, which is not part of the invention. The potentials are preferably varied in a determined way during the course of the dose forming process such that the dose obtains the intended properties. While the transfer of particles takes place from the generator through the iris diaphragm to the target area or areas of the chuck member the potential fed to the top electrode is typically a few hundred volts, positive or negative, in order to attract charged particles. The electrode on the bottom side is typically fed with a potential between zero and some tens of volts in order to slightly repel the charged particles and help guiding particles through the iris diaphragm.

The particles emerging from the aperture topside of the iris diaphragm enter the attracting field emanating from either the charges applied to the target area or areas or the electrode or electrodes behind the target area or areas of the chuck member. Combinations between pre-charging of each target area and an applied field from an electrode are also possible. The attracting electrode is typically given a potential between 500 and 2000 V. The emerging particles therefore continue on their path in the direction of the target area or areas. During the dose forming process the transfer of particles may be interrupted by the control system, which may create a strong repelling electric field within the iris diaphragm by feeding suitable opposing potentials to the electrodes such that no charged particles can penetrate the aperture of the iris diaphragm.

Further, the electric iris diaphragm may be used to screen the particles such that only small particles of preferred sizes are let through. This is achieved by superimposing an alternating AC field on the composite quasi-stationary electric field of the iris diaphragm. The working principle is based on the moment of inertia, whereby large particles have much more mass than small ones but less charge per unit weight so that the former accelerate much more slowly in a given field compared to the latter. If the frequency of the AC field is suitable, chances are that the large particles will not succeed in penetrating the iris diaphragm, since they are too heavy to oscillate sufficiently, returning to the cloud of charged powder particles as they slowly lose their charge. Finally the force of gravitation may bring them to a collection zone and back to a short-term storage of powder. These heavy particles may then be re-introduced in the process and further de-agglomerated and fed to the particle generator and re-used in the dose forming process.

Thus, in a preferred embodiment of the invention, the iris diaphragm comprises at least two electrodes separated by thin isolating wafer members between them, and at least one aperture through the iris diaphragm. The electrodes and the isolating wafer members are typically made as a printed circuit board (PCB) having a top and a bottom side. The electrode (topside by definition) closest to the chuck member is typically circular in shape and concentric with the aperture, while the other electrode (bottom-side by definition) is closest to the particle generator and may cover the lower side of the PCB completely. The chuck member is positioned upside down above the particle generator such that the net electrostatic force acting on emitted charged particles is directed upwards counteracting the force of gravity during forming of the dose. In this manner no big or heavy particles can land on the target area or areas by accident under the influence of gravity alone. This preferred positioning arrangement also helps to reduce the number of stray, charged particles from being accidentally deposited on the target area or areas. Particles such as dust or moisture exist in the atmosphere surrounding the chuck, even though the atmosphere is controlled. The force of gravity now counteracts the electrostatic force reducing the probability of unwanted depositions.

The prior art limitations in total dose mass and bad spatial control of the dose layout will be eliminated by fast and efficient neutralization of the electrostatic field created by the charged powder particles and by the target area or areas of the chuck member, i.e. the dose bed(s), thus eliminating the repelling field from the dose during forming. Very quick neutralization will be achieved, e.g. by arranging an ion-generator near the target areas of the chuck such that the emitted ions are directed towards the dose and each individual target area of the chuck member. The emitted ions ionize the air and the resulting oxygen and nitrogen ions of both positive and negative charge may be attracted to the dose and the chuck member, whereby some of them will hit the dose and the chuck member and recombine, neutralizing the accumulated charges in the process. By immediate neutralization of the particle charge once the particle has been deposited on the chuck member the negative influence from the particle charge on incoming particles is eliminated. The spatial deposition of the particles is thus vastly improved with no particles settling outside the target area or areas, because the sum of charges at the dose bed and the dose being formed as a whole is continuously neutralized in this way eliminating a distorting, repelling electric field from arising.

In a typical embodiment of the invention the accumulated charge within the dose and dose bed is regularly neutralized during the dose forming process as described. The relevant target area of the electrostatic chuck is brought within the range of an ion-generator by a servo mechanism, such that the accumulated charge is removed at least once and more preferably at least three times during the forming of the dose. It is also typical that the electrostatic chuck must pass by the ion-generator to remove any residual charge from the target area or areas before commencing a dose forming operation. Of course, the pre-charging, if used, of the individual target areas must be performed after removing residual charges. Clearly, any measurements of dose mass based on measuring of the accumulated charge from deposited particles on the target area(s) must be performed before charges are removed by the application of e.g. the ion source.

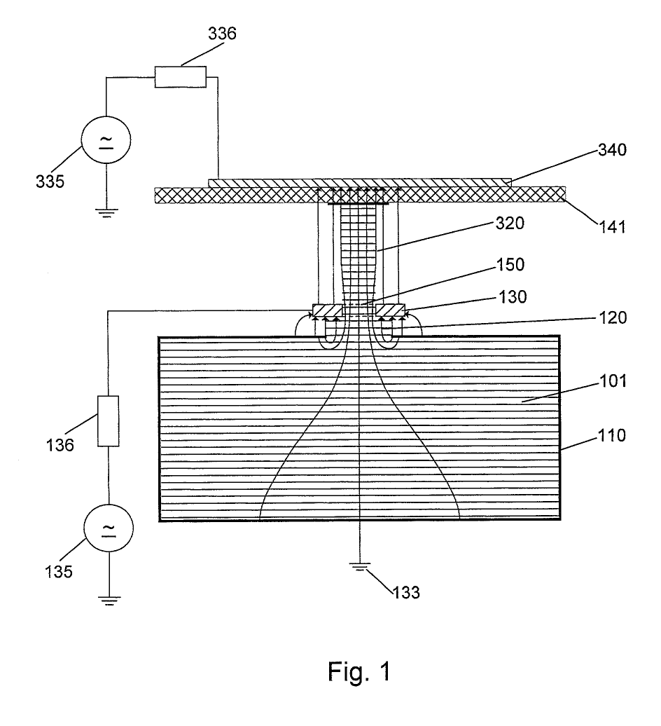

A main principle of the method according to the present invention is illustrated in FIG. 1.

The method utilizes electro-dynamic field technique in order to screen particles;

transport particles;

distribute particles over at least one pre-defined area on an electrostatic chuck;

deposit particles onto at least one pre-defined area on an electrostatic chuck;

control the mass of the dose being formed;

switch the particle flow on or off as function of time, and

control the porosity of the dose

Further, the method is based on externally applied electric fields into which the charged particles are introduced. In a preferred embodiment, electro-powder is used, but other powders may be possible to use, which is easily recognized by people of ordinary skill in the art.

The electro-powder forms an active dry powder substance or dry powder medication formulation with a fine particle fraction (FPF) presenting of the order 50% or more of the powder mass with an aerodynamic particle size below 5 μm and provides electrostatic properties with an absolute specific charge per unit mass of the order 0.1 to 25 μC/g after charging, and presents a charge decay rate constant Q₅₀ of more than 0.1 s, a tap density of less than 0.8 g/ml and a water activity a_{w} of less than 0.5.

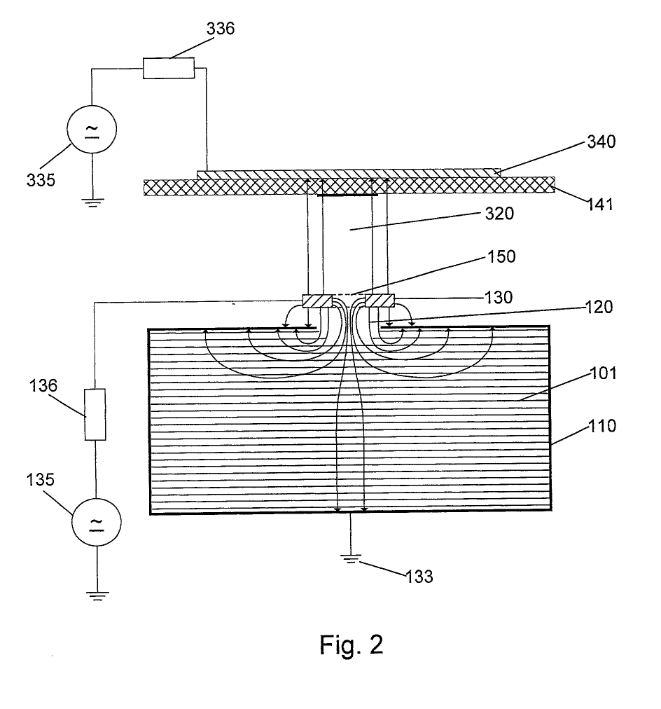

In a preferred embodiment the process will operate in an upward direction, i.e. against gravitation forces to thereby prevent particles having no charge from reaching the dose carrier in an uncontrolled way. Therefore a particle generator is positioned beneath a chuck member to carry medicament powder doses created. Referring to FIG. 1, particles 101 are released from the particle generator 110 provided with a positive or negative charge by corona-, tribo- or induction-charging, whereupon the particles enter an imposed first electric field 120. The type of charge of the particles depends on the powder characteristics, method of charging and materials in the generator so that the majority of the particles are charged either negatively or positively when they are emitted from the generator to take part in the dose forming process. In the following discussion and in the illustrations it is assumed that the emitted particles are positively charged. However, this depends on the properties of the powder and the generator and it is equally possible that the particles are negatively charged, in which case the applied potentials must change polarity, but the discussion is still valid. In order to control the dose forming process in terms of total dose mass and dose forming time, the transfer of charged particles from the particle generator to the target area or areas of the electrostatic chuck must be controlled. To this end, a first electric field 120 is applied between ground 133 and a first electrode 130 connected to a first voltage source 135, including source impedance 136. The electrode is preferably positioned a short distance in the range 0,5-25 mm from the electrostatic chuck 141 between the particle generator 110 and the chuck 141. The strength and direction of the created electric field 120 may be adjusted by adjusting the potential of the electrode within wide limits from a negative to a positive voltage, as set by the voltage source. Charged particles are thereby either attracted to (see FIG. 1) or repelled from (see FIG. 2) the first electrode, which has at least one aperture 150 of suitable size and shape where charged particles can pass through. Such apertures may be circular, elliptic, square or narrow slits or any other shape in order to suit the dose forming process. In a preferred embodiment, the aperture or apertures are in the range 50-5000 μm as main measures. However, particles attracted by the first electrode easily stick to it, which impairs the efficiency of the system and frequent cleaning may become necessary.

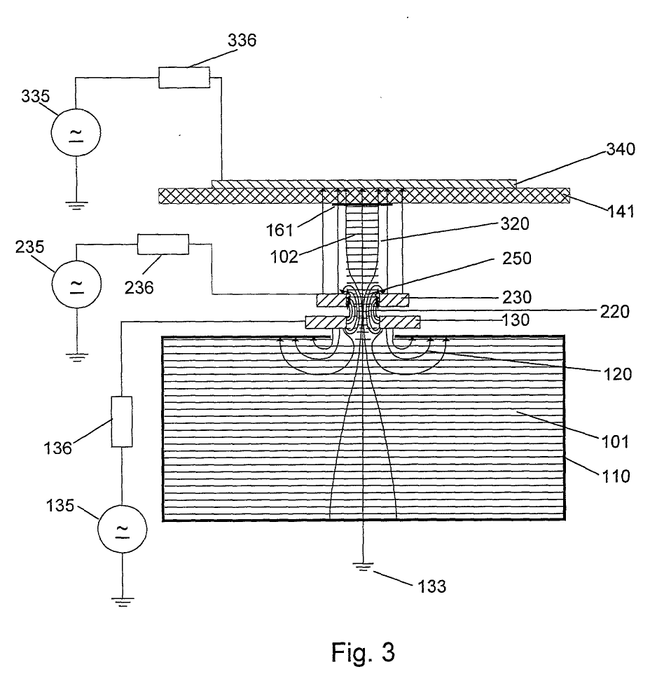

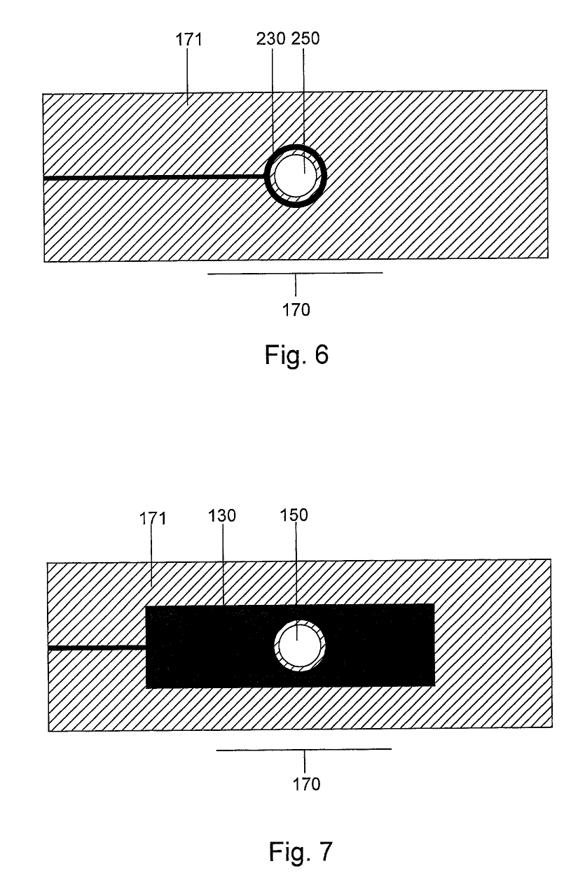

To eliminate the sticking effect and further improve the level of control of the transfer of particles to the target area or areas of the electrostatic chuck, an optional second electrode 230 as illustrated in FIG. 3 and FIG. 6, may be introduced. It should be positioned in a plane parallel to the first electrode 130, in between the first electrode and the chuck at a distance between 0,07 and 2,5 mm from the first electrode. The second electrode is perforated by the same number of apertures 250 as the first electrode by using a layout, which matches the apertures 150 of the first electrode in position and shape such that the apertures of the two electrodes are concentric. The shape and size of the electrodes may vary from very large, comparable to the target area or areas of the electrostatic chuck, to a fine circular ring less than 1 mm in diameter and less than 0,1 mm in width. Either the second electrode 230 may float electrically by not being connected to anything else or it may be connected to a second voltage source 235 with impedance 236. The strength and direction of a created second electric field 220 may be adjusted by adjusting the potential of the second electrode within wide limits from a negative to a positive voltage as set by the voltage source, if connected to the electrode. Charged particles 102 caught in the second field will travel along the field lines either in the direction of the second electrode or in the opposite direction, depending on the polarity of the applied voltage and hence the direction of the field lines.

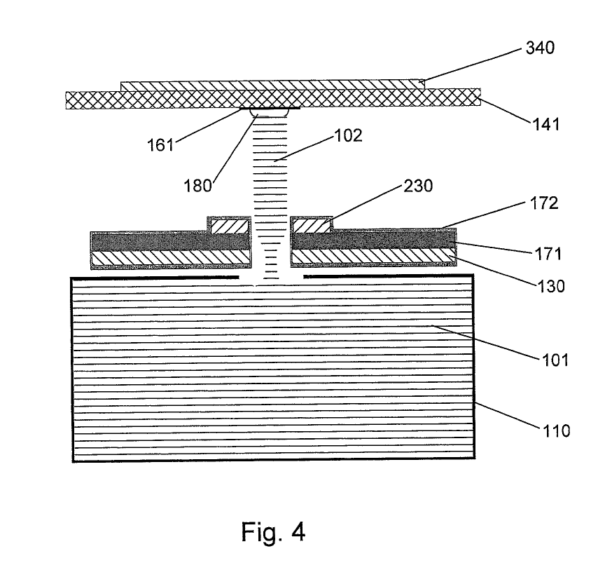

In a preferred embodiment, illustrated in FIG. 4, the first and second electrodes are integrated in an isolating wafer member 171 between the electrodes. The outward faces of the electrodes are preferably coated with an isolating coating 172 of a few microns in thickness, e.g. parylene, to stop possible short-circuiting of electrodes by sticking particles. The thickness of the wafer is typically in the range 0,07-2 mm. As an illustrative example the electrodes and the wafer member may be made as a printed circuit board. There are many types commercially available, e.g. in terms of number of possible conductor layers, physical flexibility and thickness.

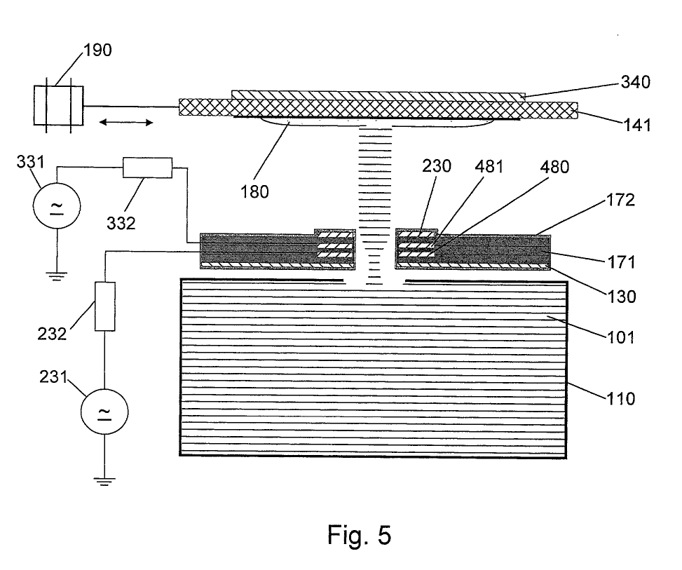

In further embodiments, as exemplified in FIG. 5, more electrodes 480, 481 may be introduced for specific purposes as, e.g. porosity control or screening of particles, which will be discussed separately. The extra electrodes 480, 481, if introduced, may be concentrically located either in extra layers of the isolating wafer member, or put in the same layer as the basic first and second electrodes. The extra electrodes are isolated from all other electrodes and ground to offer complete freedom in what connections to be made of electrodes to electric systems of controlled impedance and voltage sources. In this case the thickness of the wafer member may lie in the range 0,07-2,5 mm.

The wafer member 171 constitutes a physical barrier between the particle generator 110 and the chuck 141 with the dose bed or beds constituting the target area or areas 161 for the deposition of charged particles 102. The distance between the top electrode or electrodes on the top of the wafer member and the chuck is in the range 0,5 to 25 mm. The only possibility for the particles to reach the dose bed is therefore to go through the available apertures of the first and second electrodes and possible extra electrodes, if introduced.

A further third electric field 320 is set up between ground 133 and a third electrode 340 connected to a third voltage source 335 (see FIG. 3). It is possible to reference the third voltage source to the output of the first or second electrode instead of ground to simplify control of the deposition process. The third electrode is preferably positioned in close proximity behind the electrostatic chuck 141 and the dose bed 161, such that the electric field lines go through the dose bed in the direction of the particle generator 110. The electrostatic chuck may be made of a dielectric or semiconductive material or even a conducting material or a combination of different such materials. In the case when the material in the dose bed is conductive, the dose bed may constitute the third electrode. The strength and direction of an ensuing third electric field 320 may be adjusted by adjusting the potential of the third electrode within wide limits from a negative to a positive voltage as set by the third voltage source, if connected to the electrode, such that the charged particles are either transported towards or away from the third electrode. The electric field created by the third electrode may be combined with or replaced by the local field resulting from charges applied to the target area or areas by a charging method, e.g. corona charging. The target area or areas may be in the shape of unharmful, pharmacologically neutral beads, which are to be coated with the charged powder particles forming the dose. The beads may in some cases be pharmacologically active and they may comprise a proportion of optional excipients. There are many medication possibilities where the bead substance is favourably combined with the powder dose.

Charged particles 101 emitted from the generator 110 enter the combined electric field resulting from the potentials applied to the first, second and third electrodes respectively, the latter sometimes combined with or replaced by charges fed to the target area or areas by a source of charges of suitable polarity. The first electrode alone acts as an electric iris diaphragm device 170 and the addition of the optional second electrode improves the efficiency of the device considerably. A typical embodiment of the electric iris diaphragm is illustrated in FIGS. 6 and 7, showing the topside and bottom side respectively. It offers a possibility of controlling not only the particle transfer rate but also the apparent aperture size. The aperture or apertures through the first and second electrodes and through the isolating wafer, if present, can be made smaller or larger to the transported particles by increasing or decreasing the applied voltage potential of the first electrode while the potential of the second and third electrodes are kept constant. The electrode or electrodes, constituting the iris diaphragm, transfers charged powder particles 101, emitted from the generator, to the individual target area or areas 161 on the electrostatic chuck in a controlled orderly way in terms of mass, direction and speed, like a printer ink-jet.

In a first aspect, the electric iris diaphragm 170 controls the area of the particle stream making it possible to control the physical size of the dose as it is formed onto the target area or areas. However, in a second aspect if the first potential is increased past a certain point, the exact voltage value at this point depends mainly on the relative strengths of the first, second and third electric fields, the iris diaphragm closes so that no particles are let through at all. This offers a simple way of instantaneous starting and stopping of the particle flow and may serve the purpose of tightly controlling the distribution and deposition of particles in the process of forming a preferred electro-dose most suitable for effective system delivery by inhalation.

By adjusting the second and third potentials feeding the respective electrodes, it is possible to partly control the transfer rate of particles through the aperture or apertures in the electrodes. In this third aspect the electric iris diaphragm acts as a particle flow control valve such that it is possible to adjust the amount of particles per unit time that are let through and consequently the deposition rate on the target area.

In a fourth aspect the electric iris diaphragm may be used to screen the particles such that only small particles 102 of preferred sizes are let through. This is achieved by superimposing an AC potential of suitable frequency and amplitude from a first AC source 231, as illustrated in FIG. 5, on e.g. the quasi-stationary second potential and, if necessary, from a second ac source 331 superimpose a second ac potential synchronized with the first ac potential on the quasi-stationary third potential. Another way of adding AC fields to the quasi-stationary fields may be the adding of special electrodes 480, 481 for the purpose and integrate the new electrodes in the same wafer element as the first and second electrodes and in line with these. In this case, the AC voltages are directly applied to the new electrodes instead of superimposed to the second and/or third electrode. The physical order of the electrodes may be interchanged to optimize the screening effect. The combined effect of the quasi-stationary fields taken together with the further superimposed AC fields is to accelerate the small and light particles to the dose bed on the electrostatic chuck but exclude the big and heavy particles. The working principle is based on the moment of inertia where big particles, i.e. agglomerates, have much more mass than small ones, but less charge per unit weight so that the former accelerate much more slowly in a given electric field compared to the latter. The frequency of the AC potentials are set so that heavy particles entering the second field, controlled by the second electrode, hardly oscillate in the field while the light particles oscillate with a larger amplitude such that the third field can take control of the particle at or just before it reaches the apex of the oscillation. The strength of the third electric field will at this point overcome that of the second field and the particle breaks loose to move in the direction of the third field leaving the second field. If the frequency of the AC field is suitable, the large particles will never travel through the iris diaphragm, but will stop in the iris diaphragm until they lose their charge so that the force of gravitation can bring them to a collection zone. These particles may then be recycled and further de-agglomerated and fed to the particle generator and re-introduced in the dose forming process. In this way the electro-dynamic field technique method further reduces the number of big particles being deposited and improves the quality of the dose.

After passing the iris diaphragm 170, the particles are accelerated in the third electric field, which may have an AC component, in the direction of the target area or areas of the electrostatic chuck, i.e. the dose bed or beds 161. The transport of charged particles takes place under the influence of the attractive field force caused by the third field emanating either from the third electrode behind the dose bed or the charges supplied by a pre-charging arrangement, as discussed in the foregoing. The bed may be stationary or moving during the distribution of the particles. By utilizing a servomechanism 190, schematically illustrated in FIG. 5, the deposition of the particles can be controlled such that the spatial distribution of the particles on the dose bed area can be controlled arbitrarily.

For optimum performance when the dose 180 later is made available for inhalation, it is very important that the dose, besides consisting of small particles, also is provided with a desired porosity and structure. The porosity of the dose may be adjusted by suitably adjusting the amplitude and frequency of the second AC field superimposed on the quasi-stationary third field, which may also be adjusted suitably for the deposition process. It is also possible to adjust the porosity of the dose if the dose bed is subjected to high frequency vibration or a high frequency electric field, preferably after the distribution of particles has been completed. The porosity may be measured non-destructively by using e.g. existing, commercially available optical methods such as laser triangulation, automated image analysis or near-infrared analyzers (NIR) either during the deposition process or after the dose forming is finished. Measured data may then be used to continuously optimize the whole dose forming process on-line with the object of obtaining a dose with suitable properties, preferably meeting the specification for an electro-dose. An electro-dose is defined as electrically dosed electro-powder using electric field techniques, the dose possessing the following specification: Porosity is defined as

Dp_{electro-dose}=100−100(density_{electro-dose}/density_{electro-powder substance})>75%

In order to avoid that particles are deposited at random inside or even outside the target area or areas, because of the local repelling electric field emanating from charges of already deposited particles, the charges must be neutralized during the dose forming process. If the neutralization is successful no significant local repelling electric fields will build up, which may distort the third electric field and weaken its attractive power, which in turn may lead to a scattering of incoming charged particles. If charges accumulating in the dose(s) and dose bed(s) are frequently neutralized new particles will automatically go from the output of the iris diaphragm to the closest point of the nearest dose bed such that there is a sharp distinction between the formed doses and the surrounding areas of the chuck member.

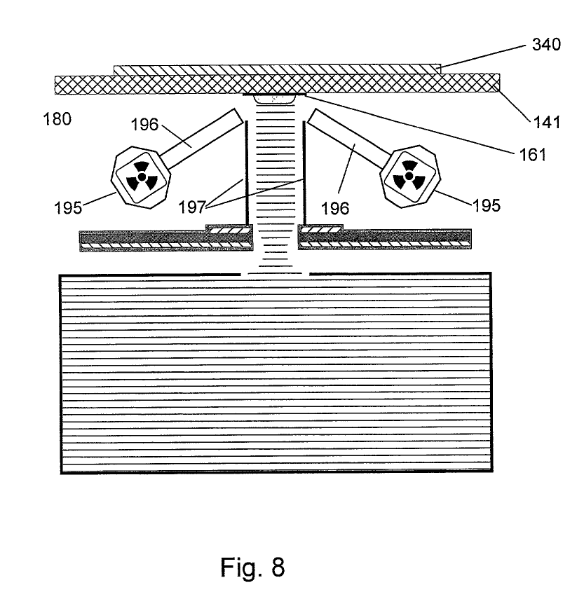

An element of the invention is schematically illustrated in FIGS. 8, 9 and 10, i.e. the element removing the accumulated charge of particles deposited on the dose bed. Various methods to neutralize charges may be used, but in a preferred embodiment a radioactive source 195 of alpha-particles (positively charged helium atoms) has been found to be most efficient. These sources are readily commercially available, e.g. from NRD LLC, Grand Island, N.Y. and are specifically used to discharge electrically charged objects. The alpha particles are scattered uniformly in all directions from a point source and ionize the surrounding air creating both positive and negative ions. The new ions are attracted to oppositely charged particles and other charged objects in the vicinity and recombine to form regular atoms using the surplus charge of the objects with which they collide. The active range from the ion source is only a few centimeters. It is very easy to stop the alpha particles within the active range by putting any solid material in the way, like a sheet of paper. A preferred radioactive point source is model P-2042 Nuclespot™, which is based on Polonium-210, but other models are available to suit all kinds of applications. Polonium-210 is currently used and has a long record of use in all kinds of industry where static electricity is a problem. The radiation leaves no residue besides helium atoms (inert gas), which are the result of the alpha particles colliding with air molecules taking up two electrons from oxygen or nitrogen atoms. In their effort to recombine, a current of ions is established that quickly neutralizes charged objects and surfaces within the active range of the radioactive point source.

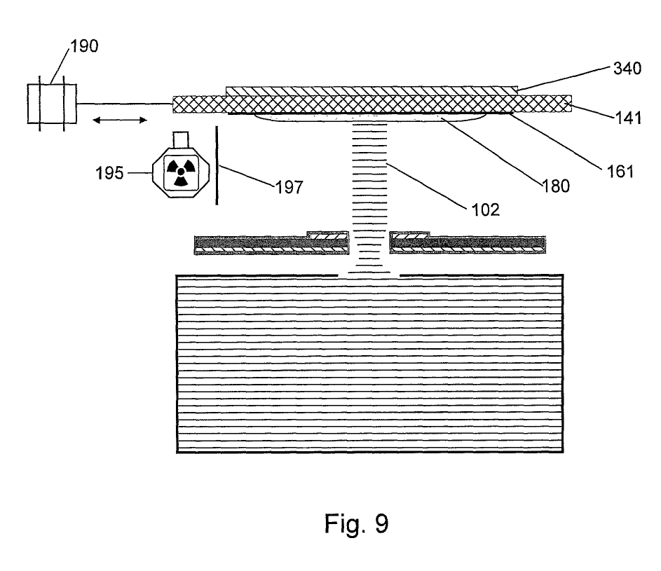

In one embodiment, illustrated in FIG. 8, it is possible to direct the alpha particles by designing at least one direction member 196 pointing to the spot on the dose bed where the powder particles 102 are deposited, such that immediately after the deposition the charge of the individual particles is removed. In a different embodiment, the ion source 195 is put outside the spot where the dose is formed, illustrated in FIG. 9. The previously mentioned servomechanism 190 is set up to withdraw the chuck member 141 with the dose bed 161 after only a partial dose forming operation before too many particles 102 have been deposited and to remove charges from the dose bed and the dose 180 by exposing the chuck member to the ion source.

For all embodiments it is generally necessary to include screens 197, which will absorb charges that otherwise risk interfering with charged particles while being transported in the electric fields set up to control the transport, distribution and final deposition of the particles in the dose forming process.

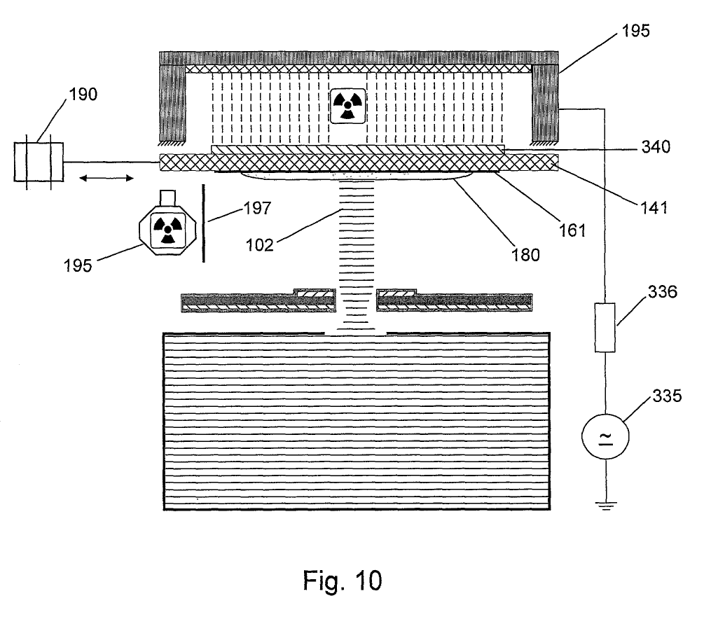

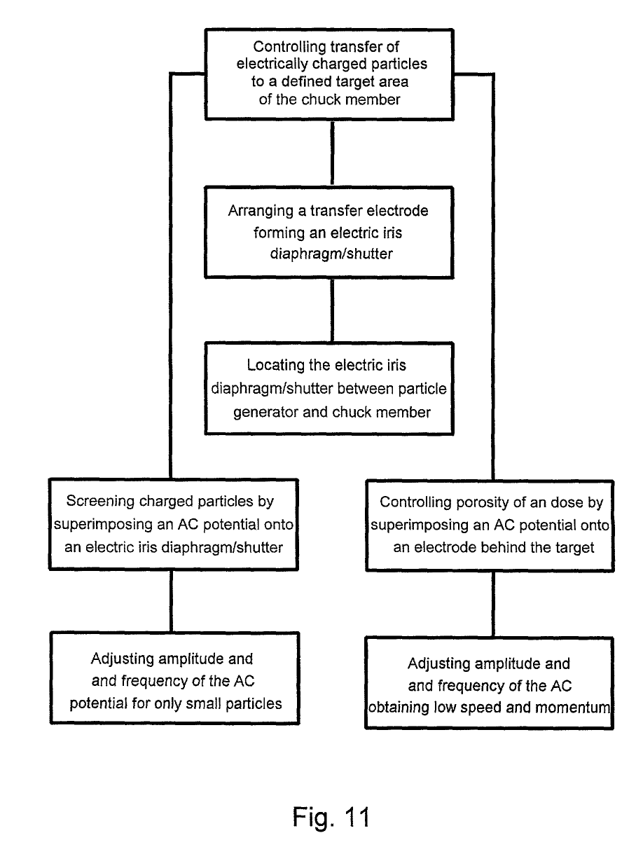

In a different embodiment physical constraints may exist in a member carrying one or more electrostatic chucks intended for doses, which make it difficult or impossible to arrange a contacting of an electrode behind the electrostatic chuck necessary for creating the third electric field as previously discussed. In such case, illustrated schematically in FIG. 10, a separate ion source 195 may advantageously be applied to make electrical contact with the third electrode 340 behind the electrostatic chuck 141 without actual physical contact. The emitted alpha particles ionize the air, which acts as an electric conductor between the ion source and the third electrode, which must be electrically conductive. The ion source should be of suitable size and placed within its working range 0-30 mm from the third electrode on the backside of the electrostatic chuck. If the metal shell of the ion source is connected to the third voltage source 335 with effective internal impedance 336, which now includes the impedance of the air gap, part of the applied voltage will also be present as a potential on the third electrode, such that the third field can be fully controlled.

It is worth noting that for all practical embodiments of the invention depositing large amounts of powder is no problem, provided the negative influence of the accumulated charge in the dose and on the chuck member is eliminated by neutralizing the charges as described in the foregoing description. Then, the field strength from the third electrode or the precharging of the dose bed is approximately constant through the developing dose. The distribution process and the forming of the dose are not sensitive to variations between particles in total charge or specific charge. As long as a particle has a charge of the right polarity and manages to pass the screening in the iris diaphragm, it will automatically be deposited onto the dose bed as long as the field exists. Provided suitable measuring instruments are put to use for monitoring the dose while it is formed, it is easy to control the described dose forming process on-line, using standard prediction, feedforward or feed-back control methods, in combination if necessary.

In a flow diagram in FIG. 11 the method of the present invention is briefly illustrated in accordance with the independent claims.

What has been said in the foregoing is by example only and many variations to the disclosed embodiments may be obvious to a person of ordinary skill in the art, without departing from the spirit and scope of the invention as defined in the appended claims.

1. A method for controlling transfer of electrically charged particles of a medication powder, intended for inhalation, emitted from a particle generator to at least one defined target are of an electrostatic chuck member in a dose forming process, comprising the steps of
arranging a particle transfer electrode member forming an electric iris diaphragm/shutter such that at least one electrode being a part of the iris diaphragm/shutter with its associated electric field operating to transfer charged particles, emitted from the particle generator, to the defined target area or areas of said electrostatic chuck member, controlling direction and speed of particles, in said dose forming process;
locating said electric iris diaphragm/shutter between said particle generator and said electrostatic chuck member such that all particles must pass the iris diaphragm/shutter in order to be transferred to the electrostatic chuck member.

2. The method according to claim 1, comprising the further step of arranging said electrostatic chuck such that its target area or areas will face downwards during the dose forming process and with the electric iris diaphragm positioned beneath this chuck in between said electrostatic chuck member and said generator of charged particles, thereby using the force of gravitation to obstruct big, heavy particles from being transferred in the created electric field from a cloud of charged particles created by the generator through the iris diaphragm to the target area or areas of the electrostatic chuck member.

3. The method according to claim 1, comprising the further step of forming an electric iris diaphragm containing an isolating wafer member and at least one electrode for controlling on one hand transfer of charged particles through the at least one aperture and on the other hand distribution of particles on one or more target areas of said electrostatic chuck member; a total thickness of said iris diaphragm being in a range of 0,07-2,5 mm, the at least one electrode having at least one aperture with a main measure in the range of 50-5000 μm; the ratio between total thickness and average aperture diameter always being less than 10 and preferably less than 3, where the average aperture diameter is defined as the sum of the two main measures of the aperture divided by two.

4. The method according to claim 3, comprising the further step of using as said iris diaphragm/shutter a flexible or rigid printed circuit board.

5. The method according to claim 1, comprising the further step of positioning said electrostatic chuck member at a distance of 0,1-5 mm from a side of said electric iris diaphragm/shutter facing the electrostatic chuck member.

6. The method according to claim 1, comprising the further step of applying quasi-stationary potentials to electrode members forming said electric iris diaphragm/shutter to switch a flow of charged particles on or off in the dose forming process.

7. The method according to claim 5, comprising the further step of applying quasi-stationary potentials to electrode members forming said electric iris diaphragm/shutter to thereby adjust a mass flow per unit time of charged particles in the dose forming process.

8. The method according to claim 1, comprising the further step of applying quasi-stationary potentials to electrode members forming said electric iris diaphragm/shutter thereby controlling the size of the aperture or apertures of the iris diaphragm/shutter setting an area of a flow stream of charged particles in the dose forming process.

9. The method according to claim 1, comprising the further step of frequently removing electrical charge from the dose or doses and the respective target area or areas of the electrostatic chuck by introducing neutralizing charges from a source member such that a repelling electric field from deposited particles is nullified.

10. The method according to any of the preceding claims, comprising the further step of using one or more ion sources to make electric contact without physical contact with one or more electrodes on a back side of said electrostatic chuck, in order to connect one or more controlled potentials to electrodes thus creating one or more necessary electric fields emanating from the electrodes for transportation of charged particles to the target area or areas in the dose forming process.

11. A method for controlling transfer of electrically charged particles of a medication powder, intended for inhalation, emitted from a particle generator to one or more defined target areas of an electrostatic chuck in a dose forming process, comprising the steps of
screening electrically charged particles of a medication powder during a dose forming process by superimposing an AC electric field onto an existing quasi-stationary field by applying an AC potential on at least one electrode of electrodes forming an electric iris diaphragm/shutter;
adjusting amplitude and frequency of said AC potential and thereby the electric field such that small, light, charged particles will oscillate in the created AC electric field, such that only small, light particles emerge from the iris diaphragm/shutter and will be transferred further in the dose forming process.

12. A method for controlling transfer of electrically charged particles of a medication powder, intended for inhalation, emitted from a particle generator to one or more defined target areas of an electrostatic chuck member in a dose forming process, comprising the steps of
controlling porosity of one or more doses of the medication powder while a dose or doses are being formed in the dose forming process by superimposing an AC electric field onto an existing quasi-stationary field by applying an AC potential on at least one electrode behind the defined target area or areas of the electrostatic chuck member where powder particles comprising a dose are to be distributed in the dose forming process;
adjusting amplitude and frequency of said AC potential such that a majority of charged particles emerging from an electric iris diaphragm/shutter are accelerated and retarded in synchronism with an AC electric field created, such that they impact on the defined target area or areas of the electrostatic chuck member with a relatively low speed and momentum resulting in an intended dose porosity.

13. A particle transfer control device for controlling the transfer of electrically charged particles of a medication powder emitted from a particle generator to one or more defined target area or areas of the electrostatic chuck member in a dose forming process, wherein
an electric iris diaphragm/shutter in a range of 0,07-2 mm in thickness, comprises at least one electrode with at least one aperture having a general measure in a range of 50-5000 μm and has ratio between total thickness and average aperture diameter always being less than approximately 10, whereby an average aperture diameter is defined as a sum of two general measures of said aperture divided by two for the purpose of bringing about electric control of on one hand transfer of charged particles through the at least one aperture and on the other hand distribution of particles onto the defined target area or areas of the electrostatic chuck member in the dose forming process;
said electrostatic chuck member having the defined target area or areas is intended for at least one pre-metered medicament dose;
an electrode behind each individual target area of said electrostatic chuck member generates a defined electric field when connected to a suitable, controlled voltage source with or without a superimposed AC voltage, such that an electric field catches and directs particles emitted from the iris diaphragm/shutter to the target area or areas of the electrostatic chuck member.

14. The device according to claim 13, wherein said electrostatic chuck is arranged such that its target area or areas will face downwards during the dose forming process and with said electric iris diaphragm positioned beneath the chuck in between said electrostatic chuck member and said generator of charged particles, thereby using a force of gravitation to obstruct big, heavy particles from being transferred in a created electric field from a cloud of charged particles created by the generator through the iris diaphragm to the target area or areas of the electrostatic chuck member.

15. The device according to claim 13, wherein said target area or areas of said electrostatic chuck member are pre-charged such that a pre-charge completely or partly in combination with an electric field from an electrode, when used, behind each individual target area creates a necessary electric field, which catches and directs particles emitted from the iris diaphragm/shutter to the target area or areas of the electrostatic chuck member.

16. The device according to claim 13, wherein quasi-stationary potentials applied to electrode members of said electric iris diaphragm/shutter create electric fields capable of switching a flow of charged particles on or off in the dose forming process.

17. The device according to claim 13, wherein quasi-stationary potentials applied to electrode members of said electric iris diaphragm/shutter create electric fields capable of controlling a mass flow per unit time of charged particles in the dose forming process.

18. The device according to claim 13, wherein quasi-stationary potentials applied to electrode members of said electric iris diaphragm/shutter create electric fields capable of controlling an apparent size of the aperture or apertures of the iris diaphragm thereby defining an area of a flow stream or flow streams of charged particles in the dose forming process.

19. The device according to claim 13, wherein electrical charge is frequently removed from formed dose or doses and corresponding target area or areas of said electrostatic chuck member by introduction of neutralizing charges from a source member such that a repelling electric field from deposited particles is nullified.

20. The device according to claim 13, wherein an ion source is used to make electric contact without physical contact with one or more electrodes on a back side of said electrostatic chuck member, in order to connect a controlled potential to its electrodes thereby creating or assisting in creating a necessary electric field emanating from the electrodes for transportation of charged particles to the target area or areas in the dose forming process.
